(19) United States
(12) Patent Application Publication
(10) Pub. No.: US 2024/0101417 A1
(43) Pub. Date: Mar. 28, 2024

(54) METHOD FOR PREPARING A SYNTHESIS GAS

(71) Applicant: Casale SA, Lugano (CH)

(72) Inventors: Ermanno FILIPPI, Castagnola (CH); Raffaele OSTUNI, Lugano (CH); Sergio PANZA, Como (CH)

(21) Appl. No.: 18/271,394

(22) PCT Filed: Jan. 20, 2022
(86) PCT No.: PCT/EP2022/051187

§ 371 Date: Jul. 7, 2023

(30) Foreign Application Priority Data
Jan. 21, 2021 (EP) 21152806.2

(51) Int. Cl. C01B 3/38; B01D 53/047; C01B 3/48; C01B 3/56; C01C 1/04; C07C 29/151

(52) U.S. Cl. CPC C01B 3/382; B01D 53/047; C01B 3/48; C01B 3/56; C01C 1/0405; C07C 29/1518; B01D 2256/16; B01D 2257/504; C01B 2203/0233; C01B 2203/0244; C01B 2203/0283; C01B 2203/042; C01B 2203/0475; C01B 2203/061; C01B 2203/068; C01B 2203/0816; C01B 2203/1235; C01B 2203/142

(57) ABSTRACT

Method for preparing a synthesis gas suitable for the synthesis of ammonia or methanol, the method comprises the step of feeding to the radiant portion of a primary reformer an oxygen-enriched air obtained by mixing air with an oxygen stream generated by water electrolysis.

### FIELD OF APPLICATION

The present invention concerns the field of syngas preparation. Specifically, the invention concerns a method for preparing a synthesis gas particularly suited for the synthesis of ammonia and methanol.

### PRIOR ART

There is a growing interest in reducing the carbon footprint of the ammonia synthesis plants.

Commercially ammonia is synthesized by treating a hydrocarbon feedstock (e.g. natural gas or coal) in a primary and in a secondary reformer to obtain a gaseous stream (syngas) comprising hydrogen, carbon oxides and impurities (e.g. methane) that after purification and compression is fed to the ammonia converter.

Steam methane reformer (SMR) and (air-blown) autothermal reformer (ATR) are two common examples of primary and secondary reforming units widely used in the ammonia industry.

SMR is a type of fired tubular steam reformer wherein a gas mixture of hydrocarbons is partially converted to syngas following an endothermic reaction between the hydrocarbons and steam. The fired tubular reformer includes at least a main radiant combustion section, heated by burners, wherein hot combustion gases obtained by combusting fuel with an oxidant indirectly exchange heat with the hydrocarbons that undergo reforming. Additionally, the fired primary reforming includes a convective section for recovery the excess of heat from the combustion gasses by means of steam superheater exchangers and water boilers. Said convective section may include additional burners for post-firing.

To further push the conversion of the uncovered hydrocarbons to syngas the partially converted gas mixture leaving the primary reformer is treated in an (air-blown) autothermal reformer.

The (air-blown) ATR comprises a partial oxidation chamber in fluid communication with a catalytic fixed bed. In the partial oxidation chamber, exothermic non-catalytic oxidation of hydrocarbons occurs generating heat that is advantageously used in the catalytic bed where the actual reforming takes place. Hydrocarbons reforming is typically carried out in the presence of steam and an oxidant, typically air or high purity oxygen separated from nitrogen in an Air Separation Unit (ASU). In the following, autothermal reforming or ATR or secondary reforming are used interchangeably.

In a plant configuration where the primary reformer is arranged in series with the secondary reformer (air-blown ATR), relatively high fuel consumption and large CO₂ emissions are expected from the combustion to sustain the reforming reactions.

Recently with the object to reduce the carbon footprint of the ammonia synthesis process, water electrolysis powered by a renewable green energy source has been envisaged for the production of so-called green hydrogen, with low carbon dioxide emissions.

WO 2019/020378 describes a process wherein green H₂ obtained by water electrolysis is fed to the ammonia converter whilst O₂ is exploited to enrich the process air fed to the secondary reformer to reduce the workload and as such the energy consumption of the air separation unit.

Unfortunately, the reduction in energy consumption and CO₂ emissions expected through this process are just apparent because several drawbacks have been noticed.

Specifically, the synthesis pressure of the O₂ obtained by water electrolysis is lower than the working pressure of the secondary reformer requiring the installation of an expensive multistage compressor and high operational cost.

Additionally, feeding oxygen-enriched air to the secondary reforming causes the temperature of the reforming gases leaving the air-blown autothermal reformer to rise. Therefore, to compensate for this effect, the temperature of the gasses leaving the primary reformer must be reduced at the expenses of a lower conversion rate of natural gas to carbon monoxide and hydrogen achievable in the primary reformer. Thus to obtain a syngas for the synthesis of ammonia, the thermal duty required for the conversion must be shifted from the primary to the secondary reformer.

Shifting the thermal duty from the primary to secondary reformer causes the carbon dioxide emissions to shift from the flue gas stack of the SMR to the CO₂ removal section of the plant, because at parity of hydrogen produced higher natural gas consumption is expected owing to the increased extent of reforming reactions in the secondary reformer compared to the primary reformer. This also entails that more natural gas added with steam must be preheated in the reformer's convection section. Additionally, a temperature reduction of the reformed gases exiting the primary reformer lowers the heat that can be effectively recovered in the reformer's convective section and reduces the temperature of the service steam that can be advantageously used to fulfil the thermal duty of the plant.

A further issue is that the oxygen obtained from the water electrolysis may fluctuate depending on the availability of the renewable sources powering the water electrolysis unit. Oxygen fluctuations may lead to additional thermal duty unbalances between the primary and the secondary reformer thus compromising the efficiency of the process. Therefore, a costly intermediate oxygen storage unit is required to compensate for this effect, usually the latter must operate at pressure higher than the conventional operating pressure of the plant.

Therefore, in light of the drawbacks set out above, it is desirable to provide a method to reduce the carbon dioxide emission while avoiding the installation of costly apparatus and avoiding thermal duty unbalances between the primary and the secondary reformer.

Methods for preparing ammonia synthesis gas are also disclosed in DE 10 2019 214 812 A1 and WO 2019/020376.

### SUMMARY OF THE INVENTION

The invention aims to overcome the above drawbacks of the prior art. In particular, the present invention seeks to provide a novel method for preparing a synthesis gas suitable for the synthesis of ammonia or methanol.

The aim is reached with a method according to claim 1.

The method comprises the step of providing a gas mixture of hydrocarbons and steam to a primary reformer reactor to yield in presence of a reforming heat a partially reformed gas or a primary reformed gas, preparing a hydrogen stream and an oxygen stream by water electrolysis, providing the primary reforming heat in the burners of a steam methane reformer (SMR) through the combustion reactions between the fuel and the oxygen-enriched air obtained by mixing air with the oxygen stream from the water electrolysis.

The term partially reformed gas denotes a gas which is reformed only partially and whose reforming is completed in a secondary reforming step, which may be autothermal reforming. The term primary reformed gas denotes a gas which is reformed in a primary reformer and is not subject to a second reforming step.

The method may comprise secondary reforming or autothermal reforming of at least a portion of the partially reformed gas obtained after primary reforming. Said secondary reforming or autothermal reforming is performed in presence of pre-heated air or in the presence of pure or substantially pure oxygen, yielding a reformed output gas enriched in hydrogen.

The term reformed output gas denotes the reformed gas which is obtained after the secondary reforming or autothermal reforming, in embodiments where secondary or autothermal reforming is performed, or after the primary reforming in embodiments where only primary reforming is performed.

The method comprises treating said reformed output gas, which is obtained directly after the primary reforming or after the secondary reforming, in one or more water gas shift sections yielding a shifted gas. The so obtained shifted gas is subjected to a further processing including at least a carbon dioxide removal step. Said further processing of the shifted gas yields therefore a CO₂-depleted gas stream and may include additional steps before or after the removal of CO₂ from the gas. Particularly, the further processing of the shifted gas may include methanation after the CO₂ removal. In some embodiments said further processing may consist of CO₂ removal followed by methanation.

In the various embodiments of the invention, at least a portion of said hydrogen stream, obtained from water electrolysis, is mixed with one or more of the following process stream: the partially reformed gas after primary reforming, before it is subjected to the secondary reforming; the reformed output gas obtained in the reforming process; the shifted gas obtained from said one or more water-gas shift sections; the CO₂-depleted gas stream obtained in the further processing of the shifted gas.

In some embodiments, the majority or the entire amount of said hydrogen stream is mixed with one of the above identified process streams. The majority of the hydrogen may be at least 60% or at least 70% or at least 80% or at least 90% of the hydrogen. In a particularly preferred embodiment, the majority or the entire amount of said hydrogen stream is mixed with the CO₂-depleted gas stream. This mixing step can be performed before or after additional treatment of the gas before or after the CO₂ removal, particularly the mixing with hydrogen can be performed before or after a methanation step.

A further object of the present invention is to revamp an ammonia plant that includes a front-end having at least one primary reforming section comprising a steam reforming portion and a radiant combustion portion, at least one shift conversion section, at least one CO₂ removal section and optionally a methanation section. The method comprises the steps of installing a water electrolysis section arranged to produce oxygen and hydrogen wherein said oxygen is fed without a compressor to said radiant combustion portion of the reforming section and optionally to said steam reforming portion of the reforming section, and at least a portion of said hydrogen is mixed with the effluent exiting the CO₂ removal section or, as an alternative, at least a portion of said hydrogen is fed to the ammonia synthesis loop through an existing compressor located after the methanation section or through a dedicate compressor if not integrated into the plant.

A further object of the present invention is to revamp a methanol or a hydrogen plant that includes a front-end having at least one primary reforming section comprising a steam reforming portion and a radiant combustion portion. The method comprises the steps of installing a water electrolysis section arranged to produce oxygen and hydrogen wherein said oxygen is fed to said radiant combustion portion and optionally to said steam reforming portion of the reforming section without a compressor, and at least a portion of said hydrogen is mixed with the effluent exiting the reforming section.

Advantageously, the method of the present invention allows a more efficient way to exploit the oxygen generated by water electrolysis. By enriching with oxygen the combustion air fed to the burners of the primary reformer, the CO₂ emissions are reduced because higher flame temperature and higher heat are developed in the radiant portion of the reformer at parity of fuel and total flow rate of oxygen combusted. Advantageously, less fuel is consumed to provide the reformer heat generated by the combustion reaction of the fuel with the oxygen-enriched air compared to the embodiment wherein the fuel is reacted with the non-enriched combustion air. Advantageously, less nitrogen is heated during the combustion reaction and the specific CO₂ emissions per unit of product, entailed by the feedstocks, and the specific CO₂ emissions per unit of product entailed by the fuel combustion in the reformer are reduced.

A further advantage of this process is that the hydrogen produced by the electrolysis of water can be exploited to increase the productivity of the plant or otherwise reducing the duty of the primary reformer having lower specific emissions of carbon dioxide.

In an embodiment, the reforming step includes a primary reforming and a secondary reforming; the primary reforming is performed at a pressure which is not greater than the pressure of the oxygen produced by water electrolysis, and said oxygen is fed directly without compression to the combustion radiant portion of the primary reforming step. Said secondary reforming may be performed in a secondary reformer or in an autothermal reformer.

According to an embodiment, said oxygen is fed directly without compression to said combustion radiant portion of the primary reformer and to said steam reforming portion of the primary reformer on the process side of the reformer.

Particularly preferably, said oxygen is fed without compression to the combustion radiant portion of the primary reformer.

In an embodiment, for example, the combustion side of the primary reforming (radiant section) operates at a pressure significantly lower than the operating pressure of the secondary reformer so that the installation of a compressor is avoided and the oxygen produced by water electrolysis can be fed directly to the primary reforming.

According to an embodiment, the operating pressure on the combustion side of the reformer is about equal to atmospheric pressure, hence even if the oxygen is obtained from the water electrolyzer at a moderate pressure, for example of 5 to 10 bar, said oxygen can be fed to the combustion side of the reformer without compression.

Preferably the pressure difference between the O₂ obtained from water electrolysis and the pressure in the combustion side is exploited for flow control purposes, for instance, it can be exploited for mixing the O₂ with the combustion air, and for passing the O₂ to the burners on the combustion side of the reformer.

The process side of the primary reformer typically operates under pressure, for example at 20 to 40 bar. Feeding the oxygen without compression to the process side of the primary reformer may be implemented when the electrolysis is configured to produce oxygen at a sufficient pressure.

A further advantage of the above-mentioned process is that the outlet temperature of the reformed gases leaving the primary reforming from the tube side may be adjusted by simply reducing the flow rate of fuel over the flow rate of the hydrocarbons that are fed to the primary reformer.

Compared to a traditional process devoid of water electrolysis, more air is fed to the secondary reformer. Hence the secondary reformer's outlet temperature may increase above a desirable value. However, according to this invention, the temperature of the secondary reformer can be advantageously controlled by reducing the outlet temperature of the steam reformer tubes.

A further advantage is that less of the combustion air must be fed to the reformer, hence the corresponding energy consumption for feeding it to the burners, and for extracting the combustion flue gas from the reformer, is reduced.

### Preferred Embodiments

Preferably when the method of the present invention is applied to the synthesis of ammonia the method further comprises the steps of treating the reformed gas in one or more water gas shift sections yielding a shifted gas, subjecting the shifted gas to a carbon dioxide removal step yielding a gas stream, mixing the CO₂ depleted gas stream with a least a portion of the hydrogen stream from the water electrolysis obtaining an adjusted make-up gas and optionally subjecting the adjusted make-up gas to a methanation reaction step yielding a purified gas stream.

Alternatively, according to another embodiment of the present invention the CO₂ depleted gas stream exiting the carbon dioxide removal step is fed directly to a methanation section yielding a purified gas stream and optionally said purified gas stream is mixed with at least a portion of the hydrogen stream from the water electrolysis.

In a particular preferred embodiment, the pre-heated air fed to the secondary reformer or autothermal reformer retains enough nitrogen to convert to ammonia the majority or the totality of the hydrogen generated in the reforming section and the hydrogen generated from the water electrolysis.

In another embodiment, a nitrogen stream is mixed with the reformed gas exiting the secondary reformer or autothermal reformer. In other embodiments said nitrogen stream can be fed to any process line located downstream the water gas shift section and prior to the ammonia converter.

Preferably, the air fed to the secondary reformer contains enough nitrogen to converter the hydrogen generated in the reforming section whilst the additional stream of nitrogen is sufficient to convert the hydrogen generated by the water electrolyzer. Preferably the nitrogen stream is obtained from an air separation unit.

According to the invention, the investment cost required to install the air separation unit and the operation cost required to carry out the fractional distillation of air are compensated in the process by the following advantages:
the air fed to the secondary reformer is not bound to provide enough nitrogen to react with the hydrogen generated in the reforming section and from the water electrolyzer; therefore, the air's flow rate fed to the secondary reformer can be adjusted to maintain the temperature of the reformed gas to the optimal value consequently avoiding thermal duty unbalance between the primary and the secondary reformer.
the ASU may be powered by renewable energy sources as such, the carbon footprint of the ammonia synthesis process is advantageously reduced.

In another preferred embodiment, the secondary reformer or autothermal reformer may be fed with pre-heated air mixed with the oxygen extracted from the air separation unit. Alternatively, the oxidant required to reform the hydrocarbons may be entirely supplied by the oxygen extracted from an ASU. According to the latter embodiment, all the nitrogen required to synthesize ammonia is preferentially provided by an air separation unit. Advantageously, the constrain to supply enough nitrogen to the ammonia converter and the necessity to limit the temperature of the gas exiting the autothermal reformer are decoupled.

Preferably, the adjusted make-up gas before being fed to the ammonia synthesis loop is treated in a methanation section to further push the conversion of carbon oxides preferably by means of a scrubber.

Preferably, when the method of the present invention is applied to the synthesis of ammonia, the pre-heated air fed to the autothermal reformer section is pre-heated in the convective portion of the reforming section.

Preferably, the method of the invention is particularly suited for the preparation of synthesis gas used for the synthesis of ammonia or methanol. Alternatively the gas may be exported and used for the other applications outside the production of ammonia and methanol for example the synthesis gas may be used as a gas combustible.

In some embodiments, a reformed output gas obtained after primary reforming is subjected to water gas shift (WGS) conversion yielding a shifted gas and to a carbon dioxide removal step preferably by means of pressure swing adsorption (PSA) unit to finally yield a CO₂ depleted gas stream. Preferably, at least a portion of the hydrogen extracted from the water electrolyser may be mixed with the shifted gas leaving the WGS section and/or with the CO₂ depleted gas stream exiting the PSA unit.

Preferably, regardless of the final application of the synthesis gas (e.g. synthesis of ammonia or methanol), the primary reforming of the mixture of hydrocarbons in presence of steam is carried out in a reforming section comprising a steam reforming portion, a combustion radiant portion and a convective portion.

Preferably, the steam reforming portion includes the reforming catalyst and is traversed by said gas mixture of hydrocarbons and steam undergoing reforming, preferably the radiant portion is configured to surround the steam reforming portion and is traversed by the oxygen-enriched air and fuel undergoing combustions.

The reforming heat may be indirectly transferred from the combustion gas towards the process gas that undergo reforming preferably from the radiant portion towards the reforming portion.

The convective portion is in fluid communication with the combustion portion and may be arranged to recover the excess of heat from the combusted gasses that is not transferred to the gasses undergoing reforming. Preferably the heat is recovered in the convective portion of the reformer by means of at least one steam superheaters and/or water boilers, other convective coils such as mixed feed gas, and process air coils may be added in the section according to the knowledge of the skilled person in the art. Heat can be recovered by way of steam production that can be exported or used in the process.

The water electrolysis can be performed by various means known in the art such as solid oxide-based electrolysis or electrolysis by alkaline cells or polymeric membrane cells (PEM). Preferably, the water electrolysis is powered by a renewable energy source consequently the corresponding CO₂ emissions are limited. Common renewable energy sources are solar energy, wind energy, hydro energy, geothermal energy, biomass energy.

According to the common knowledge of the skilled person in the art, the hydrogen stream exiting the water electrolyzer is not pure but it can contain a residual amount of oxygen in the order of a few ppb of O₂. Advantageously, when said hydrogen stream is injected prior to the methanation reactor, the residual amount of oxygen is consumed as a result of the chemical reactions occurring in said reactor.

An oxygen storage unit can be connected to the line that fed the oxygen to the primary reformer to compensate for possible oxygen production fluctuations from the water electrolyzer. Advantageously over the prior art, this oxygen storage feed tank operates at a low pressure therefore its design and its operational cost are reduced.

The method of the invention can be suitably adapted to revamp and/or to increase the production capacity of existing ammonia or methanol synthesis plants. Preferably, when the method of the invention is exploited for the preparation of a synthesis gas used for the synthesis of ammonia an air-blown autothermal reformer is used. Conversely, when the method of the invention is exploited for the preparation of synthesis gas used for the synthesis of methanol and oxygen-blown autothermal reformer is used. Preferably the oxygen fed to the oxygen-blown autothermal reformer is extracted from an air separation unit ASU. Preferably the purity of oxygen is higher than 95%, more preferably higher than 99%.

Preferably, after the installation of the water electrolysis section the volumetric flow rate of the air fed to the combustion section of the primary reformer is reduced and/or at least one heat exchanger is installed after the secondary reformer to compensate for the lack of heat recovered in the convection section of the reformer, and/or the heat recovered in the convective section of the reformer is increased by way of increasing the heat transfer surface available to recover heat in the convective section and/or at least one burner is introduced in the convective section of the primary reformer.

### DESCRIPTION OF THE FIGURES

FIG. 1 is a diagrammatic illustration of one embodiment of the present invention.

FIG. 2 is a diagrammatic illustration of another embodiment of present invention.

FIG. 3 is a diagrammatic illustration of another embodiment of present invention.

FIG. 4 is a diagrammatic illustration of an alternative embodiment of present invention.

FIG. 5 is a diagrammatic illustration of an alternative embodiment of present invention.

### DETAILED DESCRIPTION OF THE PREFERRED EMBODIMENTS

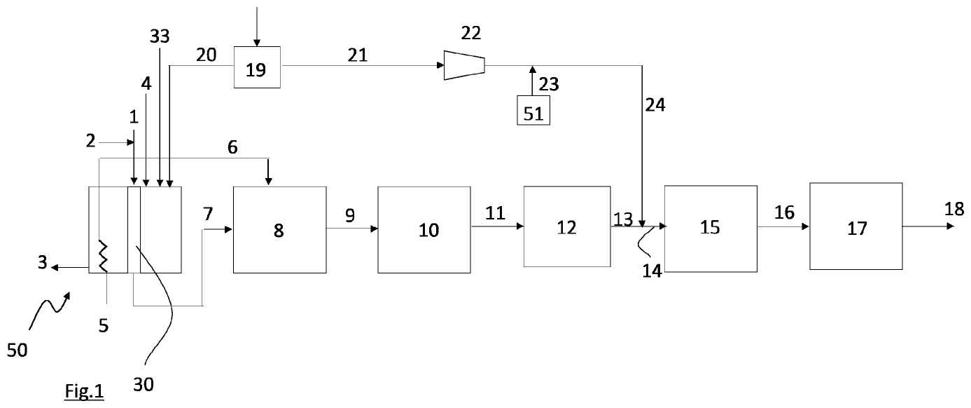

As shown in FIG. 1, fuel 4 (at ambient temperature), air 33 and oxygen 20 are supplied to the radiant combustion section of a primary reformer 50 that is heated by burners (not shown). In this section fuel 4, air 33 and oxygen 20 are oxidised realising reforming heat that is transferred to the reforming portion 51 of the primary reformer 50.

The reforming portion 30 retains the reforming catalyst is supplied with a gas mixture of hydrocarbons 1 and steam 2 that after being partially reformed are discharged through line 7.

The combustion gasses generated in the radiant combustion section of the reformer after exchanging heat with the reforming portion are subjected to heat recovery in a convective portion of the reformer 50 and are finally discharged via line 3. In the convective section of the reformer an air stream is pre-heated at the expense of the combustion gases to a temperature suitable to be fed directly to the secondary reformer 8.

The pre-heated air 6 and the partially reformed gas are fed to the secondary reformer and converted into a reformed gas that leaves the secondary reformer 8 through line 9.

The reformed gas is then fed to a water gas shift section 10 comprising a high temperature and a low-temperature Water Gas Shift WGS units, the reforming gas leaves the WGS section as a shifted gas 11 and is then fed to a CO₂ removal unit 12 (scrubber).

The CO₂-depleted gas stream 13 leaving the CO₂ removal unit is then mixed with the hydrogen 24 exiting the water hydrolysis unit 19 after compression 22 yielding an adjusted make-up gas 14. Additional hydrogen can be provided via line 23 by means of a hydrogen storage unit 51. From the water hydrolysis unit 19, oxygen 20 is extracted and fed to the reformer 50.

The adjusted make-up gas 14 is then supplied to a methanation reactor 15 for purification and then from line 16 to an ammonia synthesis loop 17. Ammonia is extracted from line 18.

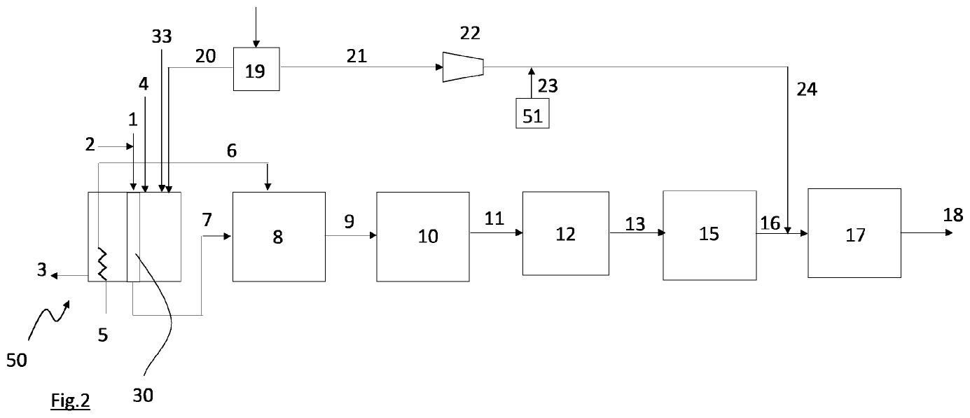

FIG. 2 shows another embodiment of the present invention wherein the hydrogen 21 extracted from the water electrolyzer 19 after suitable compression 22 is mixed with the gas effluent 16 exiting the methanation unit 15.

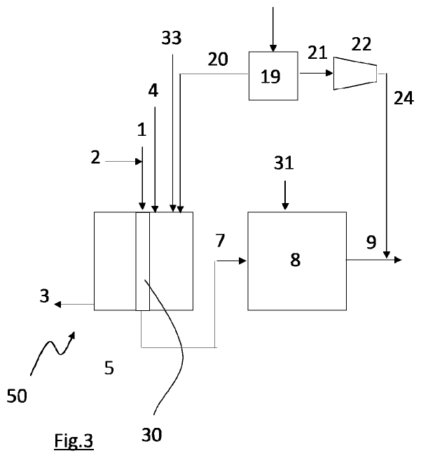

FIG. 3 shows another embodiment of the present invention wherein the autothermal reformer 8 is fed with oxygen 31 and the hydrogen 21 extracted from the water electrolyzer 19 after suitable compression 22 is mixed with the reformer gas 9 exiting the (oxygen-blown) autothermal reformer 8. The reformed gas synthetized accordingly to this configuration is particularly suited for the synthesis of methanol.

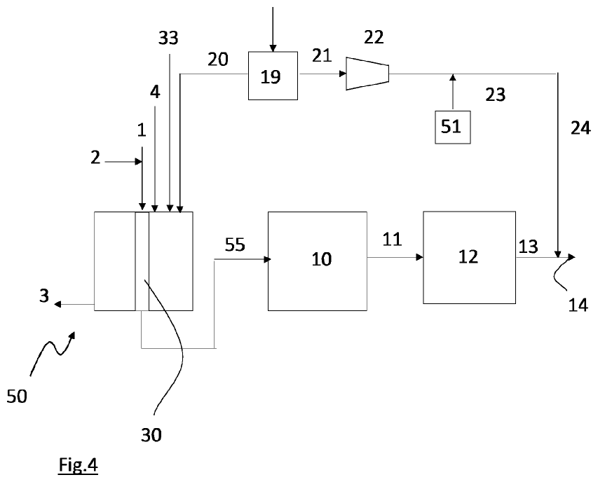

FIG. 4 shows an alternative embodiment of the present invention wherein the hydrocarbon feedstock 1 are reformed in presence of steam 2 in a primary reformer (steam reformer), no secondary reforming reactor is present. The primary reformed gas 55 exiting the reformer 50 is subjected to shift conversion in 10 and to a carbon dioxide removal in a pressure swing adsorption PSA unit in 12.

The purified gas stream 13 exiting the carbon dioxide removal reactor 12 is mixed with the hydrogen 21 exiting the water electrolyser after suitable compression in 22 to finally yield a synthesis gas 14. Additional hydrogen can be fed to line 23 through the hydrogen storage unit 51.

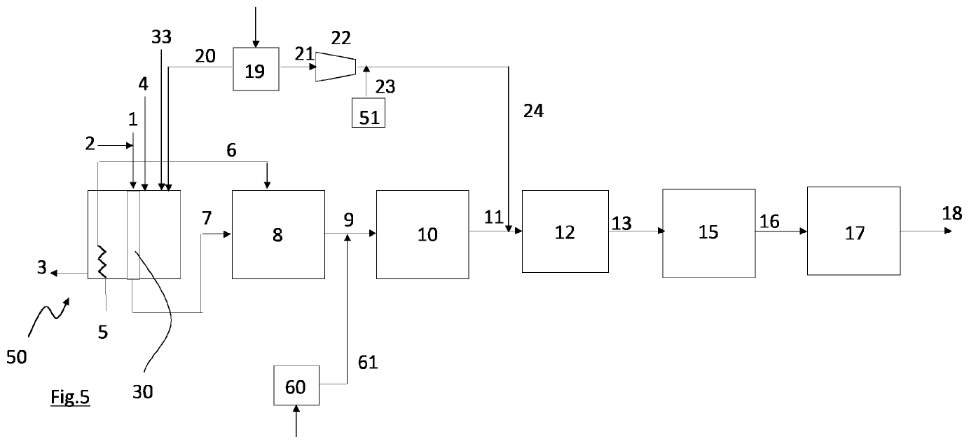

FIG. 5 shows an alternative embodiment of the present invention, wherein a nitrogen stream 61 extracted from an air separation unit 60 is mixed with the reformed gas exiting the autothermal reformer 8 through line 9.

### Example

To compare the improvements achieved by the process of this invention, the following cases have been investigated. Case 1 refers to the plant configuration wherein an air stream is fed to the secondary reformer (ATR). Case 2 refers to the embodiment of the invention shown in FIG. 1 wherein oxygen-enriched air is fed to the primary reformer (fired steam reformer).

| | Units | Case 1 | Case 2 |
|---|---|---|---|
| Production | MTD | 1016 | 1016 |
| Fuel feeds to the | kmol/h | 479 | 462 |
| primary reformer | | | |
| Oxygen feeds to the | kmol/h | 0 | 140 |
| combustion portion of | | | |
| the primary reformer | | | |
| Outlet temperature of | ° C. | 783 | 783 |
| the primary reformer | | | |
| Furnace Temperature | ° C. | 124 | 124 |
| Flue gas flow rate to | kmol/h | 6623 | 5729 |
| the stack | | | |
| Energy saving¹ | Gcal/MT | — | 0.07 |
| CO₂ emission | t/h | 20 | 19 |

¹O₂ energy not included

As it is evident from the comparison table reported above the fuel consumption is lower in Case 2 (462 kmol/h) compared to Case 1 (479 kmol/h). Analogously, the overall carbon dioxide emissions are 5% lower in Case 2 than Case 1.

1-12. (canceled)

13. A method for preparing a synthesis gas, the method comprising:
a) providing a gas mixture of hydrocarbons and steam;
b) preparing a hydrogen stream and an oxygen stream by water electrolysis;
c) a reforming process that includes at least a primary reforming of the gas mixture of hydrocarbons and steam of step a) in a presence of reforming heat and optionally includes a secondary reforming step, which is autothermal reforming, of a partially reformed gas obtained from said primary reforming, said secondary reforming being performed in the presence of pre-heated air or in the presence of oxygen, the reforming process yielding a reformed output gas which is obtained directly after the primary reforming without a secondary reforming step, or after said secondary reforming;
d) providing the reforming heat for said primary reforming of step (c) through a combustion reaction between a fuel stream and an oxygen-enriched air obtained by mixing air with the oxygen stream obtained in step b);
e) treating said reformed output gas in one or more water gas shift sections yielding a shifted gas;
f) subjecting said shifted gas to further processing including a carbon dioxide removal step, so that said further processing yields a CO₂-depleted gas stream; and
g) mixing at least a portion of the hydrogen stream of step b), obtained by water electrolysis, with at least one process stream selected from: said partially reformed gas; said reformed output gas; said shifted gas obtained from said one or more water-gas shift sections; said CO₂-depleted gas stream;
wherein the primary reforming step is carried out in a reforming section comprising a steam reforming portion, a combustion radiant portion and a convective portion; and
wherein the primary reforming step is performed at a pressure which is not greater than the pressure of the oxygen produced by water electrolysis, and said oxygen is fed directly without compression to said combustion radiant portion.

14. The method of claim 13 wherein at least a portion of said hydrogen stream, and a majority or the entire amount of said hydrogen, is mixed with the CO₂-depleted gas stream.

15. The method of claim 14 wherein the hydrogen stream is added to the CO₂-depleted gas stream before or after a methanation step of the CO₂-depleted gas.

16. The method of claim 13, further comprising mixing a nitrogen stream with the reformed output gas and/or with the CO₂ depleted gas stream.

17. The method of claim 13, wherein the synthesis makeup gas is for the synthesis of ammonia or methanol.

18. The method of claim 13: wherein
the steam reforming portion includes the reforming catalyst and is traversed by said gas mixture of hydrocarbons and steam undergoing reforming;
the combustion radiant portion being configured to surround the steam reforming portion and being traversed by said oxygen-enriched air stream of step d) and said fuel undergoing combustions;
reforming heat is indirectly transferred from the radiant portion towards the reforming portion, the convective portion being in fluid communication with the combustion radiant portion and being arranged to recover the excess of heat from the combusted gas originated by the combustion between the fuel and said oxygen-enriched air exiting said combustion radiant portion.

19. The method of claim 13, wherein reforming includes secondary reforming, wherein pre-heated air fed to the secondary reformer section or autothermal reformer section is pre-heated in the convective portion of the primary reforming section.

20. The method of claim 13, wherein said oxygen is fed directly without compression to said steam reforming portion.

21. The method of claim 13, wherein the electrolysis of water is powered by renewable energy.

22. A method of revamping a front end of an ammonia plant, said front-end being arranged to produce an adjusted make-up gas including carbon monoxide, hydrogen and residual impurities, said front-end includes at least one reforming section, at least one shift conversion section, at least one CO₂ removal section and optionally a methanation section;
the reforming section includes a steam reforming portion, a radiant combustion portion heated by burners and a convective portion in fluid communication with the radiant combustion portion, wherein said steam reforming portion is traversed by a mixture of hydrocarbons that undergoes catalytic reforming in presence of steam and reforming heat, the radiant combustion portion is traversed by a fuel that is combusted in presence of air providing said reforming heat and said convective portion is configured to recover the excessive heat generated by the combustion/oxidation reactions between the fuel and the air leaving the radiant portion of the reforming section;
the method comprising:
installing a water electrolysis section arranged to produce oxygen and hydrogen;
providing means arranged to feed said oxygen without a compressor to said radiant combustion portion of the reforming section and optionally to said steam reforming portion; and
providing means arranged to mix said hydrogen either with the CO₂-depleted gas stream exiting the CO₂ removal section or to feed said hydrogen to the ammonia synthesis loop via a dedicated compressor or via a pre-existent compressor.

23. A method of revamping a front end of a methanol or a hydrogen plant, said front-end being arranged to produce a reformed gas including carbon monoxide, hydrogen and residual impurities, said front-end includes at least one reforming section;
the reforming section includes a steam reforming portion, a radiant combustion portion heated by burners and a convective portion in fluid communication with the radiant combustion portion, wherein said steam reforming portion is traversed by a mixture of hydrocarbons that undergoes catalytic reforming in presence of steam and reforming heat, the radiant combustion portion is traversed by a fuel that is combusted in presence of air providing said reforming heat and said convective portion is configured to recover the excessive heat generated by the combustion/oxidation reactions between the fuel and the air leaving the radiant portion of the reforming section;
the method comprising:
installing a water electrolysis section arranged to produce oxygen and hydrogen;
providing means arranged to feed said oxygen, to said radiant combustion portion of the reforming section and optionally to said steam reforming portion; and
providing means arranged to mix said hydrogen with the reformed gas exiting the reforming section.

24. The method of claim 23, further comprising at least one of:
reducing the amount of the air fed to the fired combustion portion;
installing or modifying at least one heat exchanger to superheat the steam or preheat the natural gas or the natural gas mixed with steam after the secondary reformer;
increasing the heat generated in the radiant combustion portion of the reforming section; or
increasing the heat recovered in the convective portion of the reforming section.
